(19) United States
(12) Patent Application Publication
(10) Pub. No.: US 2026/0077126 A1
(43) Pub. Date: Mar. 19, 2026

(54) FLUID DELIVERY DEVICE TESTING SYSTEM AND METHOD OF USING SAME

(71) Applicant: Becton, Dickinson and Company, Franklin Lakes, NJ (US)

(72) Inventors: Danielle ABOUD, Rathgar (IE); Steve BEGUIN, Neuheim (CH); Ludovic GIL, Grenoble (FR); Sean McGrath, Dublin (IE)

(73) Assignee: Becton, Dickinson and Company, Franklin Lakes, NJ (US)

(21) Appl. No.: 18/884,429

(22) Filed: Sep. 13, 2024

(51) Int. Cl. A61M 5/168

(52) U.S. Cl. CPC A61M 5/16831; A61M 5/16886; A61M 2205/3351; A61M 2205/70

(57) ABSTRACT

An improved system and method for testing fluid delivery devices is provided. The system comprises a volumetric flow rate sensor and a pressure sensor in fluid communication with the volumetric flow rate sensor. A flow restrictor is provided downstream of the flow rate and pressure sensors. A pressure regulator controls back pressure to a collection reservoir. A processor receives flow rate signals from the volumetric flow rate sensor and pressure signals from the pressure sensors and stores the flow rate signal values and pressure signal values in a memory of the testing system.

### RELATED APPLICATIONS

### Field of the Invention

The present invention relates generally to testing systems for use with medication infusion and injection devices.

### Background of the Invention

Treatment methods for some conditions and diseases utilize a continuous delivery of a medication. One commonly used device is a medication delivery device that can be attached to the patient for delivering a continuous and controlled delivery of the medication to a patient.

A medication delivery pump can provide continuous infusion of the medication to a patient at varying rates in order to more closely match the intended treatment. Medication delivery pumps often include an infusion cannula, typically in the form of an infusion needle or a flexible catheter, that pierces the patient's skin and through which, infusion of the medication takes place. The medication pump offers the advantages of continuous delivery of a medication, precision dosing, and programmable delivery schedules.

In infusion therapy, medication doses can be administered at a basal rate and in a bolus dose. The medication pump cay also be capable of programming the basal rate of a medication to vary according to the different times of the day and night. Medication pumps may be configured to enable the patient to program the volume of the bolus dose as needed.

Conventional medication delivery pumps utilize a lead screw and piston type metering system and require a relatively large height and footprint. More recently, patch pumps have been introduced which are smaller in size, simpler in construction, and have more accurate metering mechanisms.

As new medication delivery pumps are developed, testing must be performed to ensure they operate correctly, and to check their performance under different conditions simulating use with a person. Conventionally, testing has been expensive and cumbersome, with inadequate controls. Inconsistent backpressure in conventional testing environments renders conventional testing systems suboptimal. For example, conventional testing on swine does not permit control of back pressure, and accordingly device performance in vivo is difficult to ascertain accurately.

Conventional patch pumps and medication delivery pumps typically include occlusion alarms and leak alarms. These alarms are typically based on pressure sensed by a pressure sensor (or possibly other types of sensors) in the device. Delivery pressure is a result of two main factors, resistance in the device itself, and resistance in the tissue into which fluid is delivered. The device resistance is dependent on dimensions and geometry of the device components, as well as fluid flow rate, and the possible existence of partial occlusions. Tissue resistance is a factor of the tissue structure. FIG. 4 is a cross section of a model of skin with a cannula inserted into subcutaneous tissue. In FIG. 4, skin 400 is shown to comprise an outer dermis layer 410, subcutaneous tissue 420 below the dermis 410, and muscle 430 below the subcutaneous tissue. For subcutaneous delivery devices, a cannula 440 is inserted through the dermis 410 and into the subcutaneous tissue 420 where fluid is delivered from a distal end of the cannula 440. The following equation shows how change in pressure experienced by the fluid delivery device is a factor of resistance and volumetric flow rate.

$$Δ P = R * Q \qquad \{ Equation 1 \}$$

Where ΔP is a change in pressure, R is the fluid resistance and Q is the volumetric flow rate. The fluid resistance is a function of both skin resistance and resistance derived from the device characteristics. The device resistance is dependent on fluid path tolerances and any partial occlusions that may be present. Human subcutaneous tissue pressures and resistances are known to be highly variable, and dependent on gender, age, nationality, body mass index (BMI), the injection site, among other factors. Volumetric flow rate varies from device to device, with some devices targeting, for example, ˜100 μl/min while others target, by way of example only, ˜22 μl/min. In addition, some devices deliver fluid in pulses or individual strokes, while others may deliver continuously. Some devices may have the ability to pause delivery which permits pressure dissipation as delivered fluid is absorbed into the subcutaneous tissue.

As a result of all of the variables among different pump devices, including different flow rates, pausing abilities, and device resistances, different pump designs experience different pressures during delivery of fluid.

Conventional testing methods are inadequate in that they typically hold a constant pressure regardless of volumetric flow rate, they do not allow for pressure dissipation between pump strokes and pausing, it is challenging to relate experienced pressure to a partial occlusion, and different devices have different alarm settings. Conventionally, tissue back pressure was assumed to be constant when in fact it is not. Tissue back pressure is dynamic in nature and depending on the method of pumping can lead to differing pressure profiles. For this reason, conventional testing methods using a constant back pressure throughout testing is not representative of the environment in which tested devices actually work. A set fluidic resistance (such as narrow fluidic chambers or passageways) are more representative for testing one device against another device, as different pump designs will generate different pressure profiles based on their output flow rate. Conventional systems simulate or approximate a full or complete occlusion, and thus are inadequate to test how a delivery device performs under partial occlusion. The combination of constant back pressure and full occlusion prevents the delivery device from regulating or dissipating delivery pressure, as would be expected in vivo. In vivo, a delivery device infuses causing a pressure increase, and then the delivered drug will disperse or be absorbed causing a pressure dissipation. This dissipation cannot be simulated by full occlusion or constant back pressure. Dissipation or absorption can be simulated using fluidic resistance, preferably using capillary tubing. Using capillary tubing also allows fluid flow to continue to pass through the fluid circuit and thus allows the device to have pressure dissipation between pumping strokes and/or pauses in delivery and allows devices to regulate their volumetric flow rate to reduce pressure.

Additionally, by locating a pressure regulator downstream of a fluidic resistance, such as a capillary tube, the compounding effects of fluidic resistance and a set backpressure can be more accurately ascertained.

Accordingly, there is a need for an improved testing system for medication delivery pumps. The testing system ideally addresses the above discussed inadequacies of conventional systems, and provides accurate testing to predict in vivo performance of new medication delivery pump designs, including with partial occlusions, to improve the design and evaluation process for new medication delivery pump designs.

### SUMMARY OF THE INVENTION

The above described disadvantages of conventional testing systems are overcome, and other advantages are realized by embodiments of the present application as described herein. An aspect of illustrative embodiments of the present invention is to substantially address the above and other concerns, and provide other advantages in an improved medication delivery pump testing system and method, as will be described herein.

An aspect of illustrative embodiments of the present invention is to provide an improved system for testing infusion devices. The system comprises a septum-device adapter having a recess shaped to receive a device under test, and align a cannula of the device under test with a septum in fluid communication with a fluid line of the testing system. The system further comprises a volumetric flow rate sensor in fluid communication with the septum-device adapter and a pressure sensor in fluid communication with the volumetric flow rate sensor. A flow restrictor is provided downstream of the pressure sensor. A pressure regulator controls back pressure to a collection reservoir. A processor receives flow rate signals from the volumetric flow rate sensor and pressure signals from the pressure sensors and stores the flow rate signal values and pressure signal values in a memory of the testing system.

Another aspect of illustrative embodiments of the present invention is to provide a method of testing fluid delivery devices. The method includes arranging a back pressure device downstream of a flow restrictor, weighing and filling a device under test, priming a septum of the device, selecting fluidic resistance, mechanical compliance and pressure relief parameters of the test system, volumetric aspiration parameters, filling and priming the test system, attaching the device to the primed test system, preparing pressure and flow sensors to record data, activating the device under test to begin fluid delivery from the device to the test system, completing delivery and weighing the device after the test.

Additional and/or other aspects and advantages of the present invention will be set forth in the description that follows, or will be apparent from the description, or may be learned by practice of the invention. The present invention may comprise a method or apparatus or system having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various objects, advantages and novel features of illustrative embodiments of the present invention will be more readily appreciated from the following detailed description when read in conjunction with the appended drawings, in which:

FIG. 1 illustrates a system according to a first embodiment of the present application;

FIG. 2A-2C illustrate alternate septum-device adapters according to exemplary embodiments of the present application;

FIG. 3 is a functional diagram of an exemplary testing system according to an embodiment of the present application;

FIG. 4 illustrates a model of skin tissue with an inserted cannula;

FIG. 5 illustrates a system according to a second embodiment of the present application;

FIG. 6 is a schematic diagram of a test system according to another embodiment of the present application;

FIG. 7 illustrates a stacked pressure relief valve for use with an embodiment of the present application;

FIG. 8 illustrates the principles of pressure drop in a fluid flowing through a cylindrical pipe of constant cross section;

FIG. 9 is a flowchart of an exemplary method according to an embodiment of the present application;

FIGS. 10A and 10B are charts showing exemplary data recorded during a test of a device;

FIG. 11 is a chart showing exemplary data recorded during a test of another device;

FIG. 12 illustrates pressure versus time for a syringe displacement device incorporated into an embodiment of the present application;

FIG. 13 illustrates pressure versus time for an air chamber that compresses as fluid is received;

FIG. 14 illustrates pressure versus time for a stacked pressure relief valve;

FIGS. 15A and 15B are elevation and side views, respectively, of a fluid channel of known dimensions formed in a circuit board;

FIGS. 16A and 16B illustrate a cross section of fluid tubing compressed to alter fluidic resistance;

FIGS. 17A and 17B illustrate particulate matter introduced to a filter element in the fluid path to increase fluidic resistance; and

FIG. 18 illustrates increasing pressure over time for an electronically controlled solenoid device incorporated into a testing system according to the present application.

Throughout the drawings, like reference numbers should be understood to refer to like elements, features and structures.

### DETAILED DESCRIPTION OF THE ILLUSTRATIVE EMBODIMENTS

As will be appreciated by one skilled in the art, there are numerous ways of carrying out the examples, improvements, and arrangements of a medication delivery pump testing system in accordance with embodiments of the present invention disclosed herein. Although reference will be made to the illustrative embodiments depicted in the drawings and the following descriptions, the embodiments disclosed herein are not meant to be exhaustive of the various alternative designs and embodiments that are encompassed by the disclosed invention, and those skilled in the art will readily appreciate that various modifications may be made, and various combinations can be made, without departing from the invention.

Although various persons, including, but not limited to, a patient or a healthcare professional, can operate or use illustrative embodiments of the present invention, for brevity an operator or user will be referred to as a “user” hereinafter.

Although various fluids can be employed in illustrative embodiments of the present invention, for brevity the liquid in an infusion device will be referred to as “fluid” hereinafter.

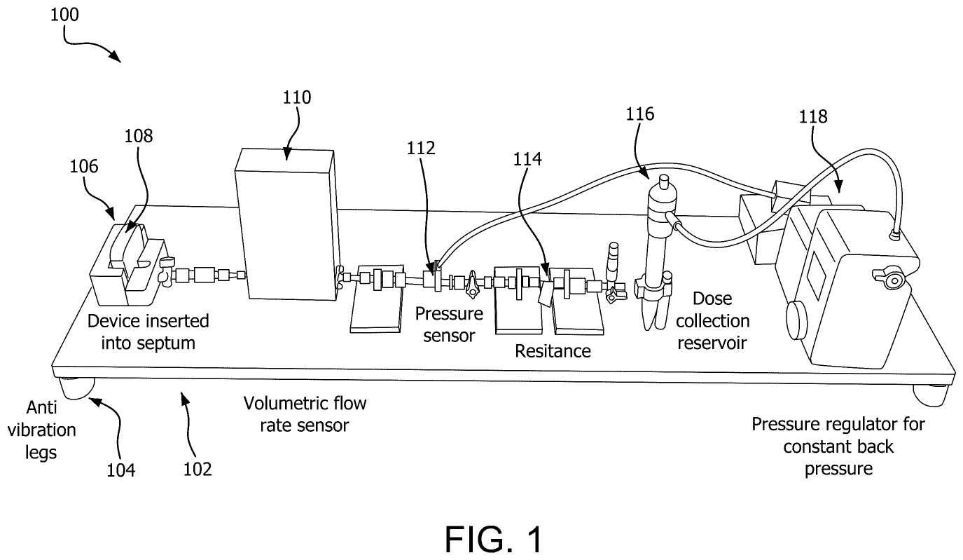

Illustrative embodiments of the present application are depicted in FIGS. 1-3 and 5-6. In an illustrative embodiment according to the present invention, a testing system is provided for use in testing infusion pumps including wearable medication infusion patches.

The testing system and method described herein simulate in vivo injection in an in vitro bench-top system. The system challenges delivery through fluidic/hydraulic resistance and/or constant back pressure in series with a drug delivery device/pump, pressure sensor(s) and volumetric flow rate sensor(s) as shown in FIG. 1. Parameters measured include: pressure, volumetric flow rate, dose delivered and delivery time. Dose delivered is preferably assessed by weighing the device under test after filling and again after delivery.

Exemplary embodiments of the present application simulate subcutaneous, intradermal and intramuscular in vivo drug delivery and as such inform design of drug delivery devices. Embodiments of this application also permit device designers to challenge, assess and optimize an injection device's delivery pressures and occlusion detection performance. Embodiments of this application also investigate the differences in device performance, and improves upon conventional testing systems that only simulate complete or full occlusion, with a pressure that is advantageously capable of dissipation due to fluidic resistance in the testing system. As a result, the test system more accurately reflects conditions when delivering fluid into subcutaneous tissue. Pressure in tissue does not remain constant, but typically peaks followed by dissipation between fluid delivery strokes. Conventional testing systems are not capable of producing or recording such dissipating pressure, and thus fall short of optimum for device performance measurement. Dissipation provides a more accurate depiction of device performance in-vivo.

Embodiments of the present application are advantageous over animal models, such as testing on swine, because they allow the control functions to be controlled and critically varied. Embodiments of the present application are also advantageous for testing spring based systems, because the testing system can measure delivery time against various in vivo fluidic conditions, and also simulate stalling of the device. This can be accomplished by testing for device empty/full criteria or indicators of the particular device under test, or by recognizing a drop off in flow rate expected as the reservoir empties. The fluids tested with a testing system according to an embodiment of the present application are not limited, and may include drugs, placebos, and solutions of specific viscosities as needed.

An exemplary testing system 100 in accordance with an embodiments of the present invention is illustrated in FIG. 1. The main components of the testing system are preferably mounted on a stable base 102. The base preferably includes anti-vibration legs 104 so that vibrations from the surrounding environment are not transferred to the testing system. A septum-device adapter 106 is provided to receive the device under test (DUT) 108. The septum-device adapter 106 is shaped with a recess to receive the DUT 108 such that the DUT 108 cannula is aligned with a septum, thereby providing fluid access to the remainder of the testing system 100 once activated. In use for testing a DUT 108 is inserted into the septum-device adapter 106 so that the DUT catheter aligns with the septum, so that when the DUT 108 is activated the septum is penetrated and a fluid connection is created between the DUT 106 and the testing system 100.

Septum-device adapter 106 is fluidly connected to a volumetric flow rate sensor 110. Volumetric flow rate sensor 110 is fluidly connected to a pressure sensor 112, which is in turn fluidly connected to a flow restrictor 114 to provide a known fluid resistance. The value of resistance of the flow restrictor 114 may of course be selected according to requirements of the test to be performed by the test system 100. The flow restrictor 114 is preferably a capillary tube of known diameter and length, but the invention is not limited to this, and any suitable flow restriction means that provides known flow resistance may be employed. Flow restrictor 114 is fluidly connected to a dose collection reservoir 116. Dose collection reservoir is in turn connected to a pressure regulator 118. The pressure regulator provides a constant back pressure to the testing system, which back pressure may be selected. Alternately, the pressure regulator may provide variable pressure to the dose collection reservoir. Such variable pressure may, for example, model a heartbeat to test under such conditions. By configuring the pressure regulator 118 downstream of the flow restrictor, the test system of embodiments of the present application is improved.

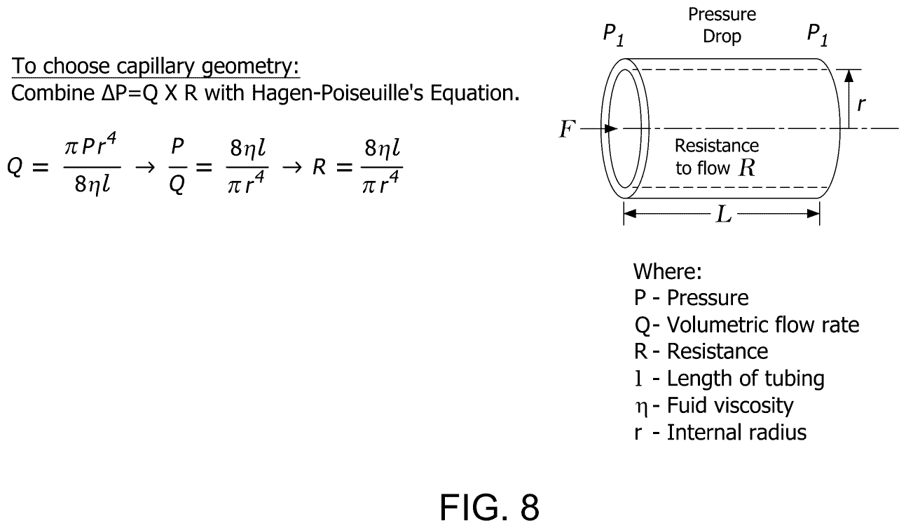

Flow restrictor 114 is preferably provided as a capillary tube of known length and radius. Fluidic resistance is controlled by the geometry (length and radius) of flow resistors/narrow capillary tubing. The pressure drop across a channel is directly proportional to the hydraulic resistance*volumetric flow rate. Combining this relationship with Hagen-Poiseuille equation, gives the following relationship describing flow resistance, which describes the pressure drop of an in-compressible Newtonian fluid in laminar flow flowing through a cylindrical pipe of constant cross section (see FIG. 8).

Using this relationship, the geometry of the capillary tubing is chosen. Using capillary tubing increases the resistance to flow within a fluidic circuit and thus increases pressure and/or reduces volumetric flow rate. The capillary tubing can allow flow to continue to pass through the circuit and thus allows the pressure to dissipate between strokes and/or pauses. This advantageously allows devices the opportunity to regulate their volumetric flow rate to reduce pressure and advantageously simulates in vivo drug dispersion and absorption. Pressure increases, which the present testing system can simulate, are expected during in vivo injection due to both delivery device characteristics, the viscosity and density of the injection fluid and properties of biological tissue (including but not limited to the local composition of the tissue and rate of absorption).

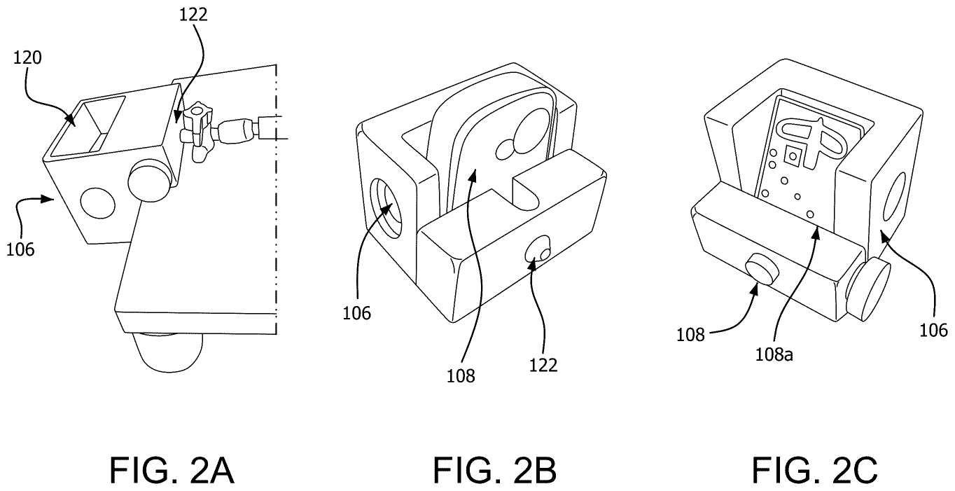

FIG. 2A illustrates a septum-device adapter 106. Septum-device adapter 106 is sized and shaped to receive a particular device under test such that the fluid delivery cannula of the device under test (not shown) is aligned with a septum through which a cannula of the device under test penetrates to make a fluid connection to the rest of the test system 100. Septum-device adapter 106 includes a device receiving opening 120 that is open to a septum 122. As illustrated in FIG. 2B, when the device under test 108 is inserted into opening 120, a cannula of the device under test 108 aligns with septum 122, after which the cannula may be deployed to create a fluid connection between the device under test 108 and the remainder of the testing system 100. FIG. 2C illustrates an alternate device under test 108a inserted into a septum-device adapter 106 shaped to accommodate the alternate device under test 108a. By modifying the dimensions of the septum-device adapter 106 practically any patch pump may be accommodated in the test system and tested.

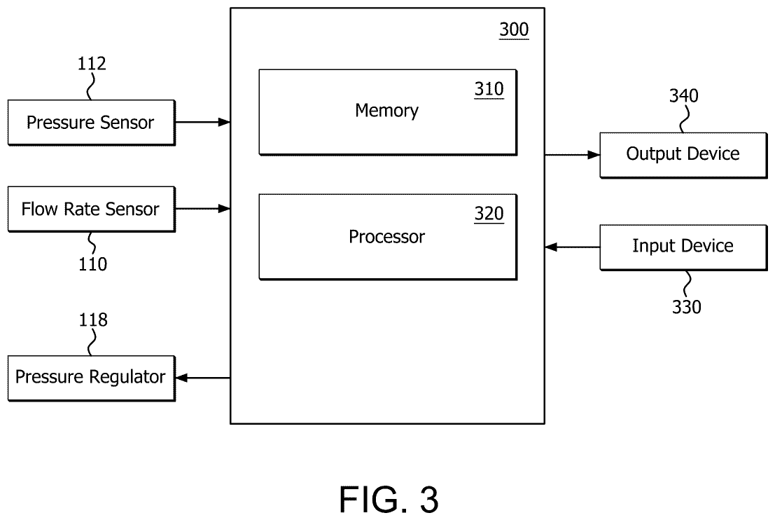

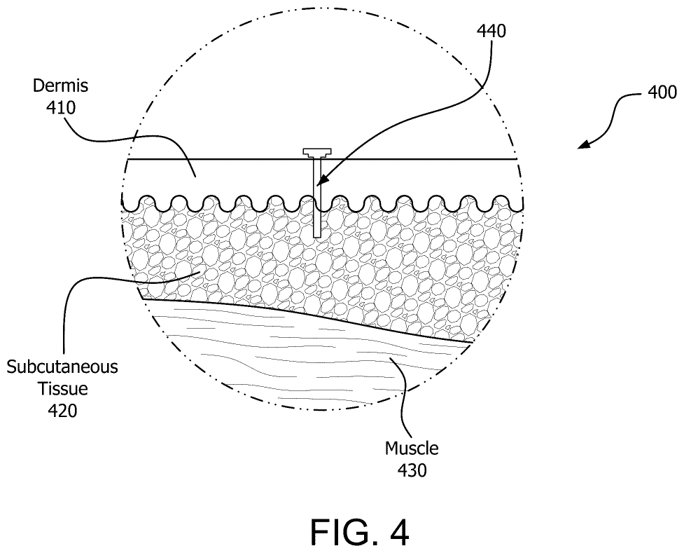

FIG. 3 is a block diagram of a computer system that forms part of the testing system 100. Computing device 300 includes a memory 310 and a processor 320. The memory 310 stores non-transitory computer executable program instructions that are executable by the processor 320. Computing device 300 is also connected to an output of the pressure sensor 112 and the flow rate sensor 110, and any other data generating elements of the testing system 100. Data output from the sensors 110, 112, etc., is preferably, but not necessarily, in digital format, and transmitted to computing device 300 for storage in memory 310 and further processing according to program instructions being run by the processor 320. Computing device 300 can also output instructions or commands to the pressure regulator 118. Computing device 300 receives input from any input device 330, including a keyboard, mouse, other input device, or any combination thereof. Computing device 300 outputs data to an output device 340 which may be a display device, printer, or any other type of output device as will be appreciated by those of ordinary skill in the art. The form of the computing device 300 and its components and input/output devices are not limited. For example, the processor 320 may be any processor capable of executing computer-executable instructions, and the memory may be a hard drive, ROM, RAM, flash memory, or any other type of non-transitory memory, or any combination of types.

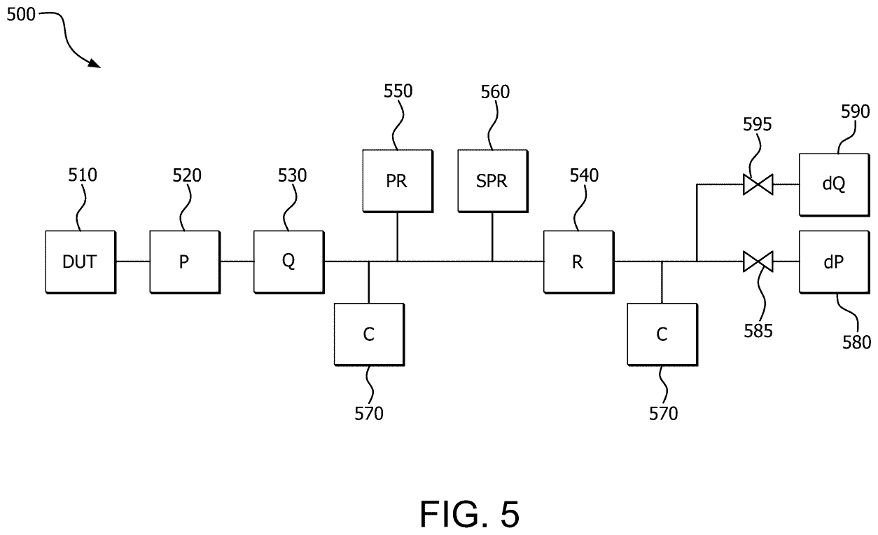

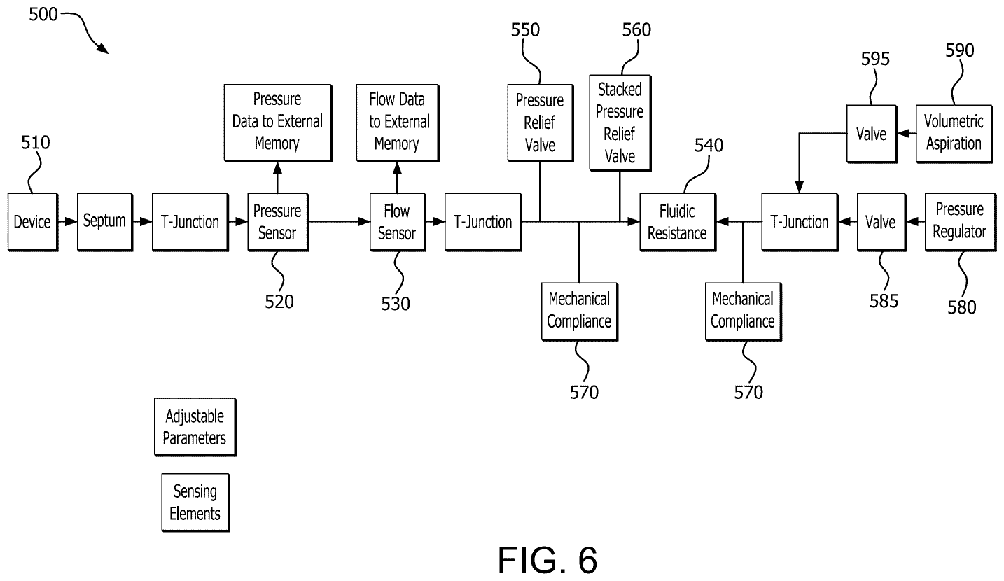

Another embodiment of the present invention is illustrated in FIGS. 5, which is a functional system diagram of system components of the exemplary test system 500, and FIG. 6, which is a schematic of exemplary physical components of the test system. As illustrated, the test system 500 comprises the device under test 510, a pressure sensor 520 and a flow sensor 530. The device under test 510 is connected to a fluid line of the system 500 via a septum-device adapter as shown in the first embodiment of FIG. 1. A flow restrictor 540 provides a selectable fluid resistance. A pressure relief valve 550 and a stacked pressure relief valve 560 are included in the system. The pressure relief valve 550 and/or a stacked pressure relief valve 560 advantageously simulate the pressure required to delaminate subcutaneous tissue, that is, the pressure at which tissue layers separate. Two mechanical compliance elements 570 are included, preferably including one mechanical compliance element 570 on the upstream side of the flow restrictor 540, and one mechanical compliance element 570 on the downstream side of the flow restrictor 540. Pressure regulator 580 is connected to the system 500 via a valve 585, and a volumetric aspiration device 590 is connected to the system 500 via a valve 595. The volumetric aspiration device 590 can be used to episodically or continually remove volumes of fluid from the test system. It should be noted that existing swine testing data, if available, can be used to inform parameters of the test system 500, such as expected tissue delamination pressure or fluid diffusion rate. Various elements of the system 500 model in vivo behavior of the device 510. For example the mechanical compliance elements 570 model varying swings in pressure magnitude. Tubing or other elements with more flexibility or compliance will deform more and accordingly have lower pressure swings with each delivery pulse, compared to stiffer elements which will deform less and have resulting higher pressure swings. The pressure relief valve 550 can model delamination of subcutaneous layers by rapidly reducing the pressure. The cutoff frequency is a device characteristic models how pulsatile flow in vivo with increasing frequency of pulses changes the pressure characteristics, such that pressure over time changes from a wave having peaks and troughs to a rise and plateau regime. That is, the flow rate changes from pulsatile to continuous as frequency increases.

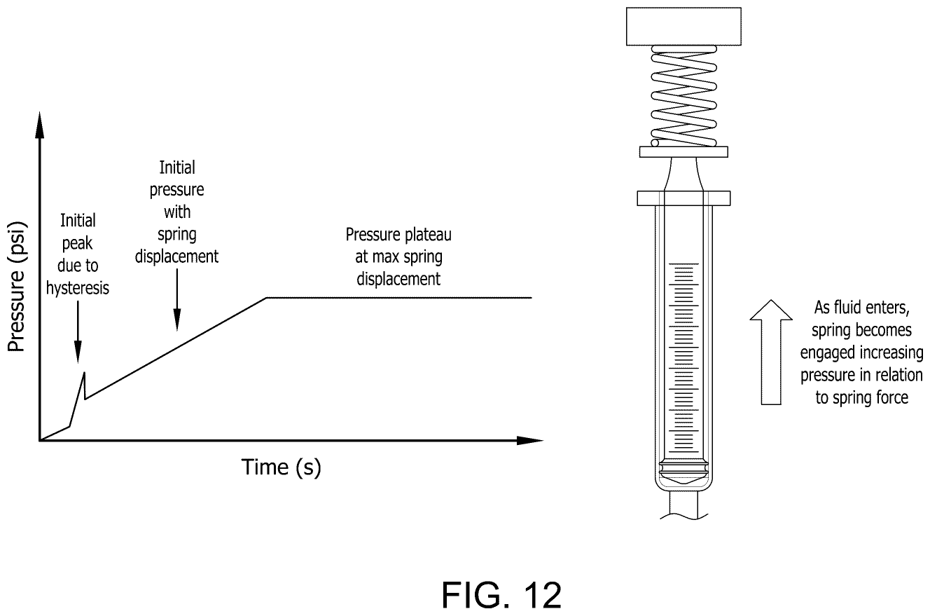

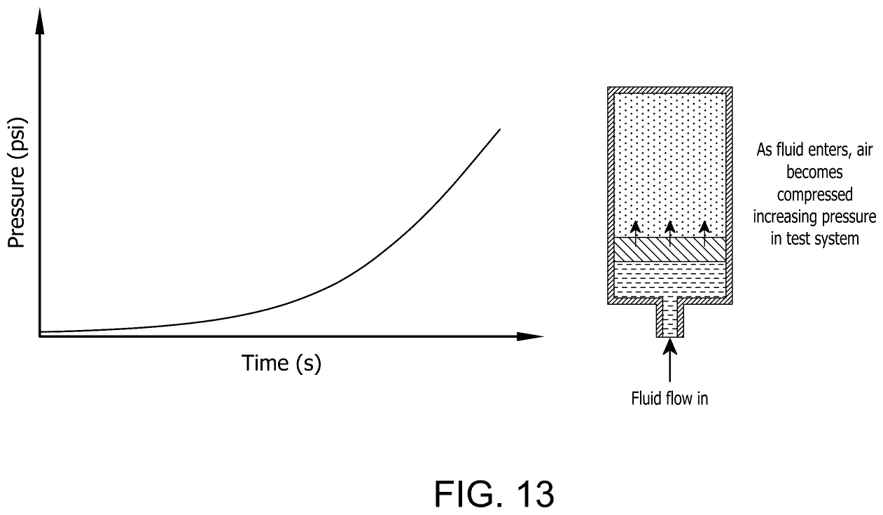

The mechanical compliance elements 570 will now be described in further detail. The mechanical compliance elements 570 model in vivo response to fluid infusion. The mechanical compliance elements 570 preferably act physically as a capacitive element acts in an electrical circuit. That is, a sum of fluid flow results in increased pressure. As a first example, a spring loaded syringe may be joined to the fluid line of the testing system at a T-junction. As fluid flows from the device under test 510 into the testing system 500 fluid lines, the spring loaded syringe is filled, resulting in increased pressure. With a mechanical spring, there may be some hysteresis due to the break loose force of the syringe, that is, the static friction between the syringe plunger and barrel. FIG. 12 illustrates pressure versus time as fluid enters a syringe and displaces a spring. A second example of the mechanical compliance element 570 is a chamber with a known volume of air. As fluid flows into the chamber, the air volume is compressed, thus increasing the pressure. FIG. 13 illustrates pressure versus time as air becomes compressed. A third example of a mechanical compliance element 570 provides tubing of known compliance. Preferably the tubing compliance and dimensions are selected to remain in an elastic deformation state throughout testing. If plastic deformation occurs, then hysteresis would be introduced to the signal. A fourth example of the mechanical compliance element 570 is a balloon that inflates as fluid flows into the balloon, with pressure increasing as the balloon is filled. A balloon can allow quasi-static behavior since the elastic modulus of the balloon is non-linear.

The testing system as configured with its components will have a cutoff frequency. Preferably the system is configured such that the system matches in vivo cutoff frequency. The flow restrictor 540 and mechanical compliance elements 570 act as a “resistor” and “capacitor”, respectively, in an analogous electrical circuit. Accordingly, by selecting values for the flow restrictor 540 and mechanical compliance elements 570 the testing system 500 can be tuned to a desired cutoff frequency. As the frequency of input pulses from the device under test 510 is increased, the pressure profile transitions from a series of peaks and troughs in pressure value to a rise and plateau.

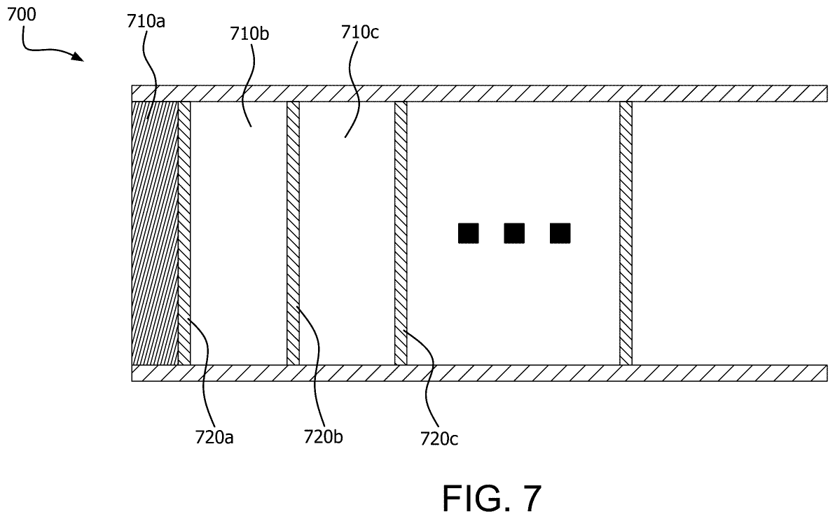

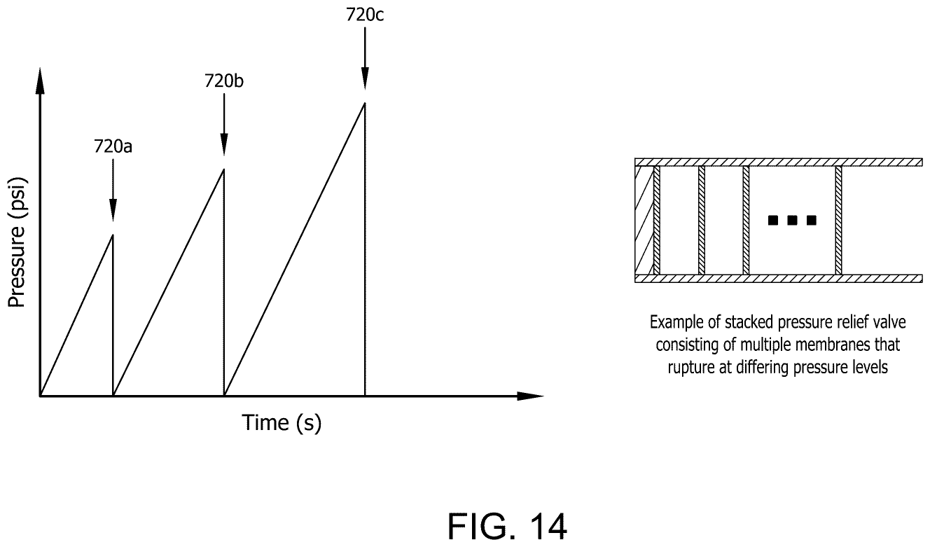

The pressure relief valve 550 models delamination of subcutaneous tissue layers as occurs when fluid pressure exceeds a yield strength of the subcutaneous tissue. The pressure relief valve is shown connected to the test system 500 at a T-junction but other configurations could be used as needed. Hydrogels of varying cross-linking levels can be used to simulate different known delamination or cracking force of subcutaneous tissue. In another embodiment the pressure relief valve is a spring loaded valve as is known. In another embodiment a stacked pressure relief valve as shown in FIG. 7 is provided. As illustrated in FIG. 7, the stacked pressure relief valve 700 comprises a series of volumes 710a, 710b, 710c, and so on, each separated by a membrane 720a, 720b, 720c, and so on, of known breakthrough force. Each cell fills with fluid until full, then when full, pressure increases until the membrane breakthrough pressure is reached and the membrane ruptures, relieving pressure, and providing access to the next cell. The cycle then repeats for as many cells and membranes are provided. Pressure versus time as membranes rupture is illustrated in FIG. 14.

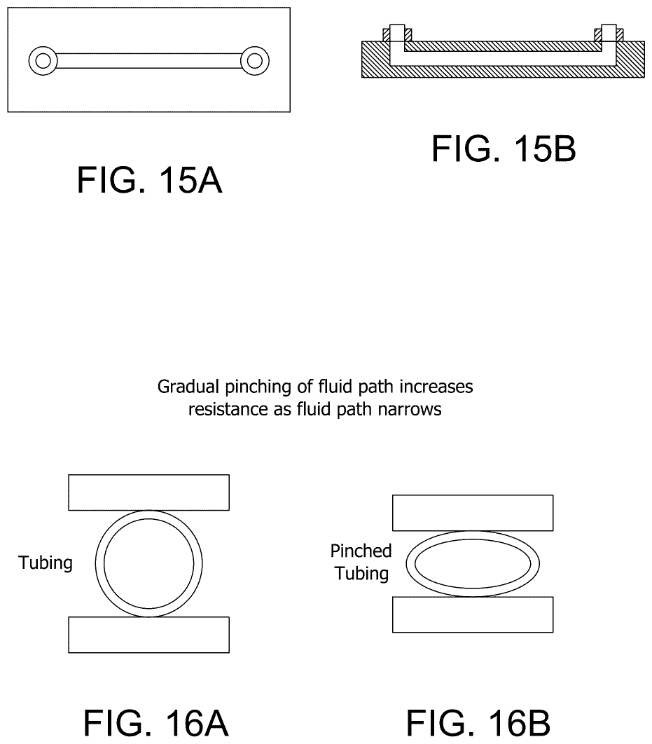

Fluidic resistance of the testing system 500 is controlled with a fluidic resistance element 540, which may be static or variable. Examples of fixed fluidic resistance elements 540 include needle valves, a higher viscosity fluid, printing narrow channels of known dimension onto a circuit board, as illustrated in FIGS. 15A and 15B, capillary tubes as previously discussed, or passing the fluid through a known porous media or membrane.

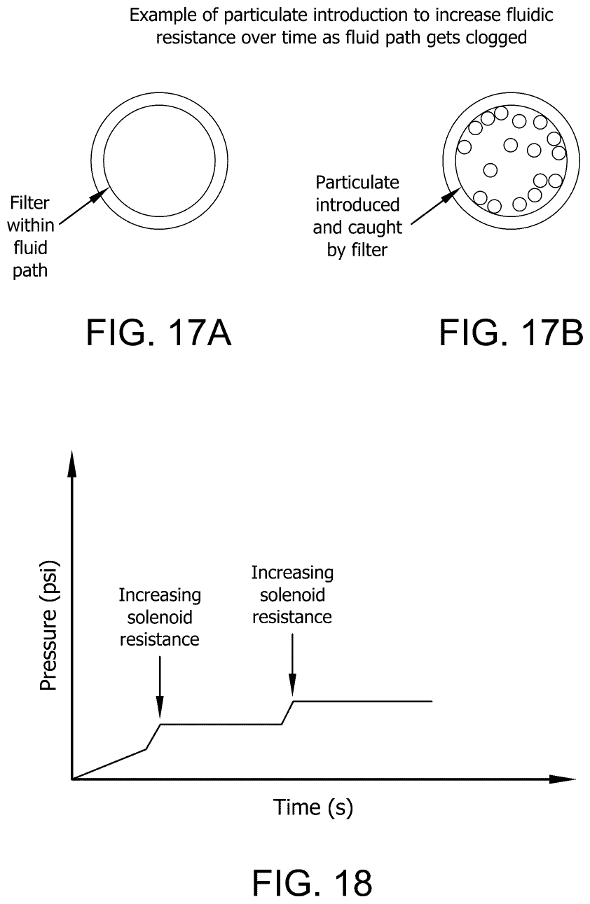

Additional testing scenarios may be simulated with a variable fluidic resistance. For example, an electronically controlled solenoid is used to vary fluidic resistance over time. FIG. 18 illustrates pressure versus time as the electronically controlled solenoid increases fluidic resistance. In another example, the fluid path diameter is continuously altered by controlling the bend of a fluid tube around a post or edge, or alternately by compressing or pinching the tube. FIGS. 16A and 16B illustrate a cross section of fluid tubing, first uncompressed (FIG. 16A), then compressed (FIG. 16B). As will be appreciated, compressing the fluid tubing increases fluidic resistance through the tubing. Deformations of these kinds advantageously simulate real world events that occur with catheters. In another example the fluid path is intentionally occluded in a controlled manner. Particulates are introduced to the testing system at a known rate and concentration, simulating aggregation during suspension delivery. In a further example, particulates are introduced upstream from a filter where they collect increasing fluid path resistance over time. Such a variable resistance advantageously simulates either solution aggregation or a biological process such as IV thrombi formation or subcutaneous catheter occlusion that increases over time, rather than happening instantaneously. FIGS. 17A and 17B illustrate a fluid path with a filter incorporated therein. FIG. 17A illustrates the filter prior to introduction of particulate matter. FIG. 17B illustrates the filter after introduction of particulate matter, which is caught in the filter, and increases fluidic resistance.

Backpressure is used in the test system to simulate the reaction of tissue to the injected fluid. As a first example, hydrostatic pressure is applied to the collection reservoir 116 by the pressure regulator 118. Hydrostatic pressure is raised by adding air to the fluid reservoir chamber through a pump or compressor, or lowered by controlled release of a valve. As a second example, hydrostatic pressure builds up as fluid fills the collection reservoir 116, thus increasing the height of fluid in the collection reservoir 116. As fluid height increases, hydrostatic pressure also increases. The shape of the reservoir can be selected to alter the rate at which hydrostatic pressure increases according to the volume of fluid delivered to the collection reservoir 116. As an example, a conical collection reservoir will initially increase fluid height at a rapid rate, and the rate will diminish as the diameter of the cone increases according to height. As a third example, the collection reservoir is a flexible reservoir, such as a flexible balloon, whose elastic properties are known.

Temperature change of the infusion fluid is another aspect that can be simulated in a testing system according to the present application. In real world settings, medication is often brought from refrigeration, at 5-8° C., or room temperature, at 23° C., and is subjected to body temperature 37° C. once injected. Change in temperature can modify the fluid's viscosity. Accordingly, a heating element such as a resistive heater may be arranged at a desired location in the testing setup to simulate temperature change of the fluid.

The volumetric aspiration element 590 models diffusion or absorption of the drug in vivo. In one example, an osmotic pump comprises osmotic membranes with check valves, simulating the lymphatic system. During in-vivo injection, at the injection site, there is a continuous exchange of solvent and chemical species between the injectate and the physiological fluids (blood and lymph). Absorption from the depot (or release in the depot) is a function of the concentration and osmolarity differences across the membranes of blood and lymph vessels. Volume aspiration elements, as described herein, reproduce the physics of uptake/release by reproducing the chemical gradients across membranes as closely as possible to in-vivo physics. In another example a porous membrane with lower fluid pressure on one side pulls fluid through the membrane. In another example, the pressure on one side of the membrane varies periodically to simulate, for example, systolic and diastolic blood pressure in the blood vessels that uptake or release chemical species at the injection site. In yet another example, a permeable membrane with adjustable chemical species concentration on either side of the membrane effectively simulates a concentration gradient in tissue such as at the leading edge of a depot or a concentration difference between the blood, the lymphatic system, or a depot. In yet another example, a membrane with known molecular weight cutoff properties simulates different diffusion rates of low molecular weight compounds compared to higher molecular weight compounds, or the effects of a higher hydrodynamic radius.

The temperature of the testing environment is optionally controlled to induce local changes in fluidic parameters including flow rate. Controlling for temperature advantageously simulates, for example, product misuse, such as drugs removed from refrigeration and used before the requisite warm-up period.

Some or all of the components of the testing system 500 may be integrated into a circuit chip, including at least the pressure sensor, flow sensor, and fluidic resistance element. Such a system on a chip is rapidly reproducible and lower in cost at volume.

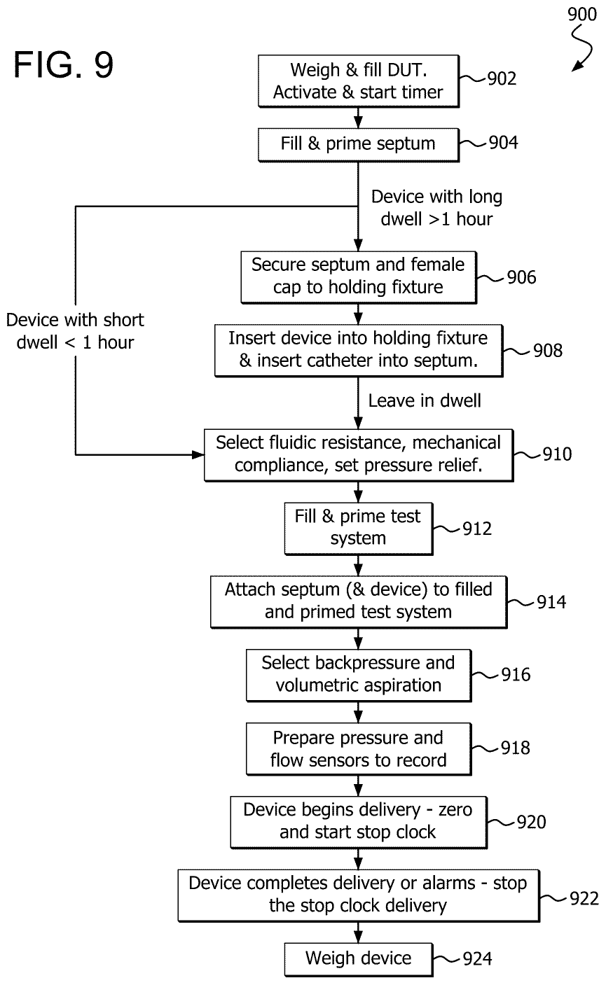

An exemplary method of testing a medication delivery device according to an embodiment of the present application will now be described in connection with FIG. 9. Implied in the method is arranging the elements of the test system as illustrated in any of the foregoing figures, including arranging the back pressure device downstream of the flow restrictor. The method 900 begins with filling and weight the device under test at step 902. At step 904 the test device septum is filled and primed. If the particular device under test is a short-dwell device (less than approximately one hour) then the method proceeds next to step 910 next. If the device under test is a long-dwell device (more than approximately one hour) then the method proceeds to step 906 next. At step 906, the device septum and female cap are secured to the device adapter. Next, at step 908, the device under test is installed into the device adapter, and the device is activated such that the device catheter is inserted into the testing system septum. At step 910 testing parameters such as fluidic resistance, mechanical compliance and pressure relief setting are selected. For fluid resistance, this may include selecting a fluid resistance element of a given length and diameter resulting in a predetermined fluidic resistance in the testing system. At step 912 the test system is filled and primed. At step 914 the device under test is attached to the filled and primed test system. The backpressure downstream of the flow resistor is selected and set, and any volumetric aspiration is selected at step 916. At step 918 the pressure and flow sensors are initialized and set to record data. At step 920 the device under test begins fluid delivery and a clock is zeroed and started. At step 922, the device under test either completes delivery of fluid or stops due to an alarm. In either event the clock is stopped and the time recorded. At step 924 the device under test is weighed and the final weight recorded. Data received from the pressure and flow sensors is also recorded for later analysis. It will be appreciated that some of the foregoing steps may be performed in different order so long as the testing method is satisfactorily completed.

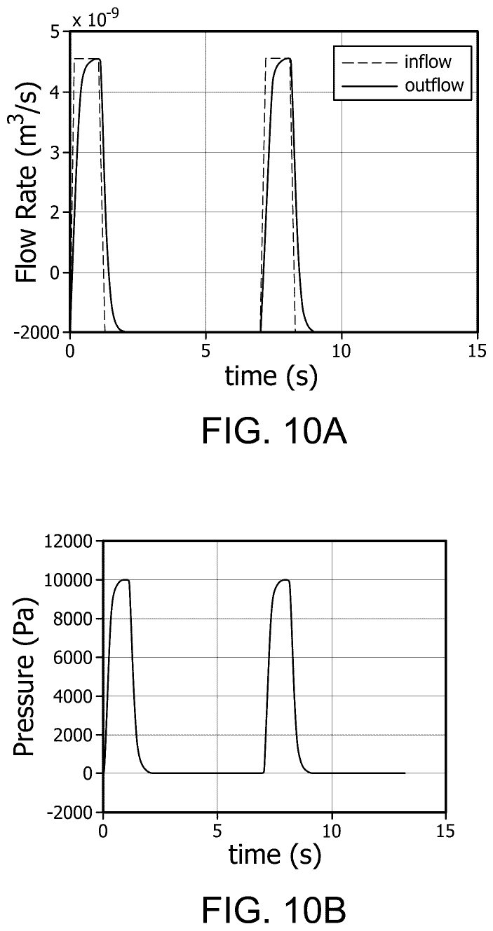

FIGS. 10A and 10B illustrate charts of exemplary data recorded during a test of a device. FIG. 10A shows flowrate over time at the test system inflow and outflow, while FIG. 10B illustrates pressure over time sensed by the pressure sensor. FIGS. 10A and 10B illustrate data recorded during a test of a device utilizing pulsatile fluid flow. Inflow flow rate shows a steep rise to a peak of approximately 4.5×10⁻⁹ m³/s, and quickly dropping to zero after approximately 1 second, the duration of a pulse. Outflow flow rate rises somewhat less quickly to a similar peak of approximately 4.5×10⁻⁹ m³/s and decays to zero after approximately 1 second when the pulse is concluded. The chart of pressure over time illustrates similar peaks and decay to zero corresponding to the delivery pulses of the device under test.

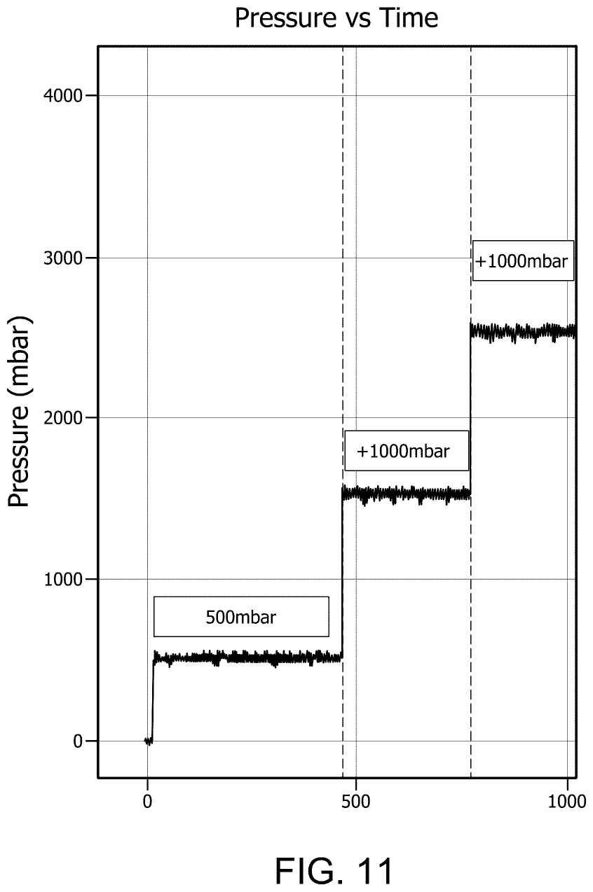

FIG. 11 illustrates data recorded from a test of another device using the test system. In this test system pressure increases with each pulse and does not dissipate. Accordingly, the pressure rises with each pulse, starting with approximately 500 mbar after the first pulse, then 1500 mbar, then 2500 mbar. In this test the device overpressure alarm would activate.

Those of ordinary skill in the art will readily appreciate that the above descriptions are merely exemplary, and are provided for a clear understanding of aspects of the present application. The positions of components of the systems described herein may be flexible, and modified to better mimic in vivo environments as needed.

What is claimed is:

1. A testing system for testing a fluid delivery device, the system comprising:
a septum-device adapter having a recess shaped to receive a device under test, and align a cannula of the device under test with a septum in fluid communication with a fluid line of the testing system;
a volumetric flow rate sensor in fluid communication with the septum-device adapter;
a pressure sensor in fluid communication with the volumetric flow rate sensor;
a flow restrictor in fluid communication with and downstream of the pressure sensor;
a collection reservoir in fluid communication with the flow restrictor and downstream of the flow restrictor;
a pressure regulator adapted to control a back pressure of the collection reservoir;
a processor; and
a memory;
wherein the processor receives flow rate signals from the volumetric flow rate sensor and stores the flow rate signal values in the memory; and wherein the processor receives pressure sensor signals from the pressure sensor and stores the pressure sensor signal values in the memory.

2. The testing system of claim 1, wherein the volumetric flow rate sensor has a fluid inlet in fluid communication with a fluid outlet of the septum-device adapter.

3. The testing system of claim 1, further comprising a base and anti-vibration legs supporting the base, wherein at least the septum-device adapter, the flow restrictor, and the collection reservoir are supported on the base.

4. The testing system of claim 1, wherein the flow restrictor has a constant fluid resistance of predetermined value.

5. The testing system of claim 1, wherein the flow restrictor has a variable fluid resistance.

6. The testing system of claim 5, wherein the flow restrictor comprises an electronically controlled solenoid.

7. The testing system of claim 1, wherein the flow restrictor is a capillary tube with a predetermined diameter and length.

8. The testing system of claim 1, further comprising a pressure relief valve in fluid connection with the testing system upstream of the flow restrictor.

9. The testing system of claim 1, further comprising a stacked pressure relief valve comprising a plurality of cells of known volume separated by membranes of known breakthrough pressure.

10. The testing system of claim 1, further comprising a mechanical compliance element.

11. The testing system of claim 10, wherein the mechanical compliance element comprises a spring loaded syringe in fluid communication with the testing system at a t-junction such that a system pressure increases as the spring loaded syringe fills from fluid dispensed by the device under test.

12. The testing system of claim 10, wherein the mechanical compliance element comprises a chamber of air in fluid communication with the testing system.

13. The testing system of claim 10, wherein the mechanical compliance element comprises tubing of predetermined compliance.

14. The testing system of claim 10, wherein the mechanical compliance element comprises a balloon in fluid communication with the testing system that is inflated by fluid flowing from the device under test.

15. The testing system of claim 1, further comprising a volumetric aspiration device in fluid communication with the flow restrictor, downstream of the flow restrictor, via a valve.

16. The testing system of claim 1, wherein the pressure regulator is electronically controlled.

17. A method of testing a fluid delivery device, comprising the steps of:
filling the fluid delivery device with fluid;
weighing the filled fluid delivery device;
priming a septum of the fluid delivery device;
selecting a fluidic resistance of a fluid resistance element of a testing system and connecting the fluid resistance element to the testing system downstream of a pressure sensor;
connecting the fluid delivery device to an input of the testing system;
priming the testing system;
selecting a backpressure of a pressure regulator connected to the testing system downstream of the fluid resistance element;
activate the fluid delivery device to begin fluid delivery by the fluid delivery device into the testing system;
after the fluid delivery device completes fluid delivery, weighing the fluid delivery device.

18. The method of testing of claim 17, wherein, if a dwell time of the fluid delivery device is greater than one hour, the method further comprises the steps of:
securing the fluid delivery device to a holding fixture of the testing system,
securing the septum of the fluid delivery device to the holding fixture, and
inserting a catheter of the testing system into the septum.

19. The method of testing of claim 17, further comprising the step of setting a volumetric aspiration volume of a volumetric aspiration device of the testing system.

20. The method of testing of claim 17, further comprising the step of recording pressure and flow signals from respective pressure and flow sensors arranged upstream of the fluid resistance element of the testing system.
